(19) United States
(12) Patent Application Publication
(10) Pub. No.: US 2003/0212085 A1
(43) Pub. Date: Nov. 13, 2003

(54) Treatment of fibromyalgia and chronic fatigue syndrome

(76) Inventors: Robert B. McCall, Kalamazoo, MI (US); Robert Clyde Marshall, Mattawan, MI (US)

Correspondence Address: Austin W Zhang, Pharmacia & Upjohn Company, Global Intellectual Property, 301 Henrietta Street, Kalamazoo, MI, 49001, US

(21) Appl. No.: 10/258,203

(22) PCT Filed: Apr. 17, 2001
(86) PCT No.: PCT/US01/10806

(51) Int. Cl. A61K 31/519; A61K 31/48; A61K 31/4745

(52) U.S. Cl. 514/267; 514/288; 514/292; 514/295

(57) ABSTRACT

The present invention relates to the treatment of neuromuscular disorders and, more specifically, to the use of apomorphine, bromocriptine, pergolide, ropinirole, octahydropyrazolo[3,4-g]quinolines, and trans-(±)-substituted-5,5a,6,7,8,9-,9a,10-octahydropyrimido[4,5g]quinolines, and their pharmaceutically acceptable salts to treat, or to prepare a medicament for treating, symptoms of fibromyalgia syndrome and chronic fatigue syndrome.

### FIELD OF THE INVENTION

The present invention relates to the treatment of neuromuscular disorders and, more specifically, to the use of apomorphine, bromocriptine, pergolide, ropinirole, octahydropyrazolo[3,4-g]quinolines, and trans-(±)-substituted-5,5a,6,7,8,9-,9a, 10-octahydropyrimido[4,5-g]quinolines, and their pharmaceutically acceptable salts to treat, or to prepare a medicament for treating, symptoms of fibromyalgia syndrome and chronic fatigue syndrome.

### BACKGROUND OF THE INVENTION

Chronic fatigue syndrome (CFS), also referred to as chronic fatigue immune disorders, chronic fatigue immune disorders syndrome, yuppie flu, fatigue-chronic, and chronic fatigue and immune dysfunction syndrome, is a clinically defined condition characterized by profound tiredness or fatigue. In addition, patients with CFS generally report various nonspecific symptoms, including weakness, muscle aches and pains, excessive sleep, malaise, fever, sore throat, tender lymph nodes, impaired memory and/or mental concentration, insomnia, and depression. The exact cause of CFS is unknown and, to date, there are no specific tests to confirm the diagnosis of CFS, though a variety of tests are usually done to exclude other possible causes of the symptoms.

Fibromyalgia syndrome (FMS), also referred to as fibromyalgia, fibromyositis, fibrositis, or myofasica pain syndrome, is a rheumatic condition generally characterized by widespread pain in fibrous tissues, muscles, tendons, and other connective tissues, fatigue, headaches, lack of restorative sleep, and numbness. Thus, FMS shares many clinical features with CFS. Similar to CFS, there are no specific diagnostic tests for FMS. Because of the similarity in clinical features between CFS and FMS, CFS and FMS are often treated similarly.

Many medications are commonly used to treat CFS and FMS. Examples of the more common medications include hypnotics, immune suppressants, various other prescribed medications, and an array of non-prescription medications. Examples of other prescription drugs include opioid antagonists, sodium retention agents/beta blockers, calcium channel blockers/histamine blockers, anti-depressants, allergy medications, and acute anxiety medications. However, there are no known medications that permanently resolve the symptoms of either CFS or FMS. In addition, many of the currently used medications produce side effects ranging from mild side effects, e.g., drowsiness, dizziness, and nausea to serious side effects, e.g., addiction and liver damage.

Accordingly, there is clearly a need for better treatments for chronic fatigue syndrome and fibromyalgia. Now, the present invention reveals several compounds that can be used to treat, or to prepare a medicament for treating, these conditions. These compounds include apomorphine, bromocriptine, pergolide, ropinirole, octahydropyrazolo[3,4-g]quinolines, and trans-(±)-substituted-5,5a,6,7,8,9-,9a,10-octahydropyrimido[4,5-g]quinolines, and their pharmaceutically acceptable salts.

U.S. Pat. No. 3,717,639 discloses apomorphine and methods of preparation of the same. U.S. Pat. No. 5,756,483 discloses intranasal formulations for apomorphine and the use of the same as a therapy for Parkinson disease. U.S. Pat. No. 5,939,094 discloses transdermal formulations comprising apomorphine. U.S. Pat. No. 6,200,983 discloses oral formulations for apomorphine and the use of apomorphine as a therapy for human sexual dysfunction.

U.S. Pat. No. 3,752,814 discloses bromocriptine and the process of making the same. U.S. Pat. No. 3,752,888 discloses information on the formulations, dose levels and administration for inhibiting lactation, as well as process of synthesis, for bromocriptine. U.S. Pat. No. 5,679,685 discloses information on the formulations, dose levels and administration of bromocriptine for inhibiting lactation.

U.S. Pat. No. 4,166,182 discloses the preparation of pergolide and its oral or parenteral administration as a prolactin inhibitor and in the treatment of Parkinson's Disease. German patent application DE 4240798 discloses a pharmaceutical composition containing ergot derivatives, including pergolide, for oral, sublingual, parenteral, percutaneous or nasal administration. U.S. Pat. No. 6,001,390 discloses a pharmaceutical composition containing ergot derivatives, including pergolide, for transdermal administration.

U.S. Pat. Nos. 4,452,808 and 5,336,781 disclose ropinirole and methods for preparing ropinirole and its pharmaceutically acceptable salts. U.S. Pat. Nos. 4,452,808 and 4,912,126 discloses methods for preparing pharmaceutical compositions for oral, rectal, or parenteral administration of ropinirole as a treatment for cardiovascular disorders or depression. European Patent Application No. 299602A discloses the use of ropinirole as a treatment for Parkinson's disease, as well as pharmaceutical compositions for oral, rectal, parenteral, or transdermal administration of ropinirole. U.S. Pat. No. 5,807,570 discloses transdermal formulations and administration of ropinirole.

U.S. Pat. No. 4,198,415 discloses octahydropyrazolo[3,4-g]quinolines and method for preparing the compounds and pharmaceutical compositions thereof for oral or parenteral administration for treating Parkinsonism and for inhibiting prolactin secretion. U.S. Pat. No. 4,528,290 discloses the formulations for oral and parenteral administration for octahydropyrazolo[3,4-g]quinolines.

U.S. Pat. No. 4,501,890 discloses trans-(±)-substituted-5,5a,6,7,8,9-,9a,10-octahydropyrimido[4,5-g]quinolines and method for preparing the same, as well formulations for oral or parenteral administration for treating Parkinson's syndrome, sexual dysfunction, depression, hypertension, and elevated prolactin levels. U.S. Pat. No. 4,521,421 discloses information on method of preparation and formulations of trans-(±)-substituted-5,5a,6,7,8,9-,9a, 10-octahydropyrimido[4,5-g]quinolines for oral and parenteral administration for treating sexual dysfunction.

### SUMMARY OF THE INVENTION

The invention is directed to the use of certain known compounds to treat, or prepare medicaments for treating, symptoms of fibromyalgia syndrome and chronic fatigue syndrome, two debilitating conditions for which no known effective treatments exist. The compounds described herein are apomorphine, bromocriptine, pergolide, ropinirole, octahydropyrazolo[3,4-g]quinolines, and trans-(±)-substituted-5,5a,6,7,8,9-,9a,10-octahydropyrimido[4,5-g]quinolines, and their pharmaceutically acceptable salts. The preparation of medicaments, dosages and routes of administration are all provided to the extent needed to identify and treat patients in need thereof.

### DETAILED DESCRIPTION OF THE INVENTION

The present invention provides for the use of several classes of compounds, and their pharmaceutically acceptable salts to treat, or prepare medicaments for treating, symptoms of fibromyalgia (FMS) or chronic fatigue syndrome (CPS). “Pharmaceutically acceptable” as used throughout the entire document refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. By “treat” or “treating” it is meant to make less hard to bear, reduce or decrease, or lighten or relieve patients of the symptoms of FMS or CFS.

A suitable compound in the present invention is apomorphine and a pharmaceutically acceptable salt thereof. Apomorphine, or (R)-5,6,6a,7-Tetrahydro-6-methyl-4H-dibenzo[de,g]quinoline-10,11-diol or 6aβ-aporphine-10,11-diol, can be structurally represented by formula (I)

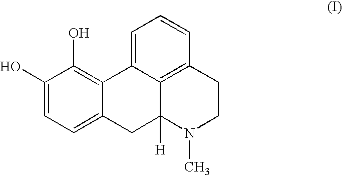

and exists in a free base form or as an acid addition salt.

The method of synthesis and preparation of pharmaceutical formulations for apomorphine, as well as its use as a therapy for Parkinson disease or human sexual dysfunction, is known in the art. See, e.g., U.S. Pat. Nos. 3,717,639, 5,756,483, 5,939,094, and 6,200,983, all incorporated herein by reference.

For the purposes of the present invention, a pharmaceutical composition comprising apomorphine or a pharmaceutically acceptable salt thereof, in a pharmaceutically acceptable carrier can be administered. Apomorphine hydrochloride is preferred; however, other pharmacologically acceptable moieties thereof can be utilized as well. In addition to the hydrochloride salt, other acceptable acid addition salts are the hydrobromide, the hydroiodide, the bisulfate, the phosphate, the acid phosphate, the lactate, the citrate, the tartarate, the salicylate, the succinate, the maleate, the gluconate, and the like.

Apomorphine can be prepared in any conventional dosage form such as a capsule, tablet, chewable tablet, powder, transdermal patch, suspension or solution. A typical formulation for apomorphine comprises 0.25 to 10 milligrams (mg) of the active and as inactive ingredients, microcrystalline cellulose, anhydrous dibasic calcium phosphate, croscarmellose sodium, magnesium stearate, hydroxypropylmethylcellulose, titanium dioxide, lactose, triacetin, and FD&C Blue #2 aluminum lake.

Apomorphine in the known appropriate pharmaceutical dosage form for a given route of administration may be administered orally, sublingually, buccally, intranasally, transdermally, or parenterally. It is preferred that apomorphine be administered parenterally by subcutaneous injection, intravenous injection or infusion, or by transdermal delivery.

The effective dosage of apomorphine is generally from about 0.25 to about 6 mg/dose/person, once or twice per day. The exact dose to be administered and the dose frequency of apomorphine should be easily determined by the attending physician or clinician.

Another suitable compound in the present invention is bromocriptine, or a pharmaceutically acceptable salt thereof Bromocriptine, or (5′α)-2-Bromo-12′-hydroxy-2′-(1-methylethyl)-5′-(2-methylpropyl)ergotaman-3′,6′,18-trione; 2-broinoergocryptine; or 2-bromo-α-ergokryptin, can be structurally represented by formula (II) below.

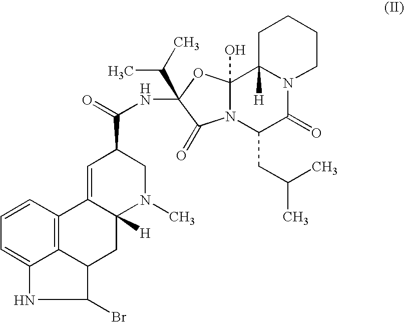

The method of synthesis and preparation of pharmaceutical formulations for bromocriptine, as well as its use and administration for inhibiting lactation, is known in the art. See, e.g., U.S. Pat. Nos. 3,752,814, 3,752,888, and 5,679,685, all incorporated herein by reference.

Bromocriptine may be obtained by brominating ergocryptine in an inert solvent, with a mild brominating agent, e.g. N-bromophthalimide, and purifying the resulting bromocriptine in manner known per se. Bromocriptine can be prepared in any conventional dosage form such as a capsule, tablet, chewable tablet, powder, suspension or solution. Solid oral preparations have long been commercially available as bromocriptine mesylate (PARLODEL® marketed by Novartis Pharmaceuticals Corp.) in tablet and capsule form containing 2.5 and 5 mg of bromocriptine, respectively. These preparations are approved in the United States for use in the treatment of certain hyperprolactinemia-associated dysfunctions and acromegaly, in the prevention of physiological lactation, and in the treatment of Parkinson's disease and prevention of tolerance to Levodopa therapy for Parkinson's disease.

Bromocriptine in the known appropriate pharmaceutical dosage form for a given route of administration may be administered orally, sublingually, buccally, transdermally, or parenterally. It is preferred that bromocriptine be administered orally.

The therapeutic effective amount of bromocriptine in the present invention is generally from about 2.5 to about 15 mg/day/person. Normally, administration of bromocriptine should be initiated at a low daily dosage of about 1.25 mg per day, and on an individual basis, the dosage is increased slowly and gradually until an optimal therapeutic response is achieved. The exact dose to be administered and the dose frequency for bromocriptine should be easily determined by the attending physician or clinician.

Another suitable compound in the present invention is pergolide, or a pharmaceutically acceptable salt thereof. Pergolide, or (8β)-8-[(Methylthio)methyl]-6-propylergoline; D-6-n-propyl-8β-methylmercaptomethylergoline; or LY-141B, can be structurally represented by formula (III) below.

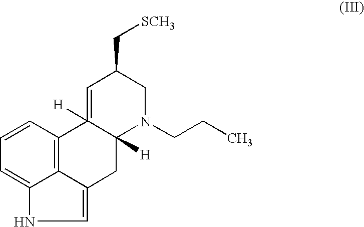

The method of synthesis and preparation of pharmaceutical formulations for pergolide, as well as its use and administration as a therapy for Parkinson's disease or as a prolactin inhibitor, is known in the art. See, e.g., U.S. Pat. Nos. 4,166,182 and 6,001,390, and German patent application DE 4240798, all incorporated herein by reference.

Pergolide can be prepared in any conventional dosage form such as a capsule, tablet, chewable tablet, powder, suspension or solution. Solid oral pergolide preparations have long been commercially available as pergolide mesylate (PERMAX® by Athena Neurocsiences) in tablet form containing 0.05, 0.25, and 1 mg of pergolide, respectively. These preparations are approved in the United States for use in the treatment of Parkinson's disease.

Pergolide in the known appropriate pharmaceutical dosage form for a given route of administration may be administered orally, sublingually, buccally, transdermally, nasally, or parenterally. It is preferred that pergolide be administered orally and parenterally, and more preferred that pergolide be administered orally.

The therapeutic effective amount of pergolide in the present invention is generally from about 0.75 to about 5 mg/day/person. Normally, administration of pergolide should be initiated with a daily dosage of 0.05 mg for the first two days. The dosage should then be gradually increased by 0.1-0.25 mg/day every third day until an optimal therapeutic dosage is achieved. Pergolide is usually administered in divided doses 3 times per day. The exact dose to be administered and the dose frequency for pergolide should be easily determined by the attending physician or clinician.

Another suitable compound in the present invention is ropinirole or a pharmaceutically acceptable salt thereof Ropinirole, or 4-[2-(Dipropylamino)ethyl]-1,3-dihydro-2H-indol-2-one; 4-[2-(di-n-propylamino)ethyl]-2(3H)-indolone; or 4-(2-di-n-propylaminoethyl)-2-(3H)-indolone, can be structurally represented by formula (IV) below.

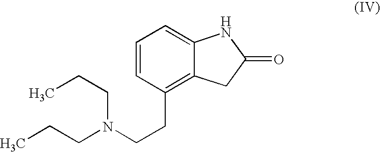

Suitable salts will be apparent to those skilled in the art and include, for example acid addition salts, preferably the hydrochloride.

Ropinirole and its pharmaceutically acceptable salts can be prepared by the methods described in U.S. Pat. No. 4,452,808. Information on formulations and administration of ropinirole as a treatment for cardiovascular disorders or Parkinson's disease is described in U.S. Pat. Nos. 4,912126 and 5,807,570, and EP-299602-A, all incorporated herein by reference.

Ropinirole can be prepared in any conventional dosage form such as a capsule, tablet, chewable tablet, powder, transdermal patch, suspension or solution. Solid oral ropinirole preparations have been commercially available as ropinirole hydrochloride (REQUIP by SmithKline Beecham) in tablet form containing 0.25, 1 mg, 2 mg, 4 mg, and 5 mg of ropinirole, respectively. These preparations are approved in the United States for use in the treatment of Parkinson's disease.

Ropinirole in the known appropriate pharmaceutical dosage form for a given route of administration may be administered orally, sublingually, transdermally, rectally, or parenterally. It is preferred that ropinirole be administered orally and parenterally, and more preferred that ropinirole be administered orally.

The therapeutic effective amount of ropinirole in the present invention is generally from about 0.25 to about 25 mg/day/person. It is typically given 3 times daily. The dosage is normally initiated at a low dosage at about 0.25 mg three times daily, and increased gradually to achieve a maximum therapeutic effect, balanced against the principal side effects of nausea, dizziness, somnolence and dyskinesia. The exact dose to be administered and the dose frequency should be easily determined by the attending physician or clinician.

Other suitable compounds in the present invention are those generically or specifically disclosed in U.S. Pat. Nos. 4,198,415 and 4,528,290. These compounds are generically referred to as trans-(±)-octahydro pyrazolo[3,4-g]quinolines and can exist as tautomers of formula (Va)

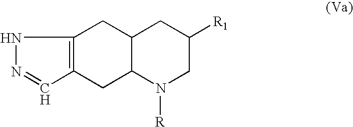

and formula (Vb)

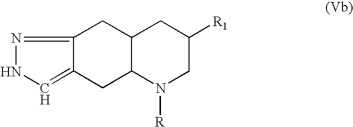

or their pharmaceutically acceptable salts, wherein:

R is H, CN, C1-C3 alkyl, allyl or benzyl and

R1 is H, COOH, COO(C1-C3) alkyl or CH2 X wherein X is CN, Cl, I, Br, OH, OCH3, SCH3, SO2 CH3, OSO2--(C1-C.sub.3)--alkyl, O--SO2--tolyl, OSO2-phenyl or CONH2.

Examples of compounds of formula (Va or Vb) include, but are not limited to, following compounds:

trans-(±)-5-n-propyl-4,4a,5,6,7,8,8a,9-octahydro-1H(and 2H)-pyrazolo[3,4-g]quinoline and dihydrochloride salts thereof;

trans-(±)-5-n-propyl-7-methylmercaptomethyl-4,4a,5,6,7,8,8a,9-octahydro-1H(and 2H)-pyrazolo[3,4-g]quinoline and dihydrochloride salts thereof;

trans-(±)-5-(C1-C3)alkyl (or allyl)-7-(C1-C3)alkoxy carbonyl-4,4a,5,6,7,8,5a,9-octahydro-1H(and 2H)-pyrazolo[3,4-g]quinoline;

trans-(±)-5-n-propyl-7-ethoxycarbonyl-4,4a,5,6,7,8,8a,9-octahydro-1H(and 2H)-pyrazolo[3,4-g]quinoline; and

trans-(±)-5-methyl-7-ethoxycarbonyl-4,4a,5,6,7,8,8a,9-octahydro-1H(and 2H)-pyrazolo[3,4-g]quinoline.

A particularly preferred compound of formulae (Va) and (Vb) can have the formula

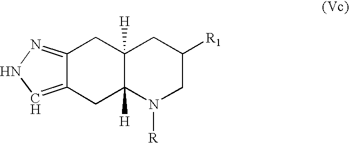

and formula (Vd)

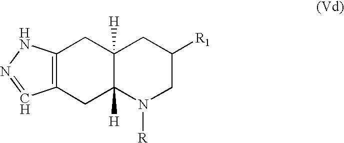

or a pharmaceutically acceptable salt thereof, wherein:

R is C1-C3 alkyl or allyl, and

R1 is CH2 X wherein X is CN, CONH2, SCH3, SO2 CH3 and OCH3.

Another particularly preferred compound of formulae (Va) and (Vb) is a trans-(+) stereoisomer existing as a tautomeric pair of the formula (Ve)

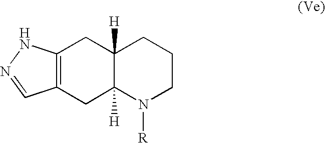

and formula (Vf)

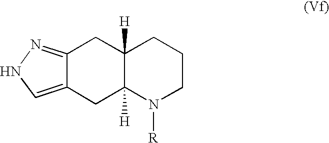

wherein R is methyl, ethyl, n-propyl, or allyl.

An especially preferred compound of formulae (Va) and (Vb) is quinpirole, or pharmaceutically acceptable salts thereof. Quinpirole, or 1H-Pyrazolo[3,4-g]quinoline, 4,4a,5,6,7,8,8a,9-octahydro-5-propyl-, (4aR,8aR)-(9CI) or 1H-Pyrazolo[3,4-g]quinoline, 4,4a,5,6,7,8,8a,9-octahydro-5-propyl-, (4aR-trans)-; (−)-Quinpirole; LY 156258, can be structurally represented by the formula (Vg) below.

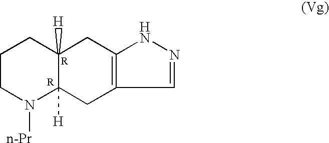

Compounds of formulae (Va) and (Vb) can be prepared by any suitable methods. Methods for preparing these compounds are further described in U.S. Pat. Nos. 4,198,415 and 4,528,290, both incorporated herein by reference. Compounds of formulae (Ia) and (Ib) can be prepared in any conventional dosage form such as a capsule, tablet, chewable tablet, powder, transdermal patch, suspension or solution. Pharmaceutical formulations for these compounds are further described in U.S. Pat. Nos. 4,198,415 and 4,528,290, both incorporated herein by reference.

A compound of formulae (Va) and (Vb) in the known appropriate pharmaceutical dosage form for a given route of administration may be administered orally, sublingually, transdermally, or parenterally. It is preferred that the compound be administered orally and parenterally.

The therapeutic effective amount of a compound of formulae (Va) and (Vb) in the present invention is generally from about 0.01 to about 15 mg/kg/dose by oral administration and from about 0.0025 to about 2.5 mg/kg/dose by parenteral administration. The oral dosage is typically given 3-4 times per day, giving a daily dosage range of about 0.3 to about 60 mg/kg per day. The exact dose to be administered and the dose frequency should be easily determined by the attending physician or clinician.

Still other suitable compounds in the present invention are trans-(±)-racemates of the formula (VI)

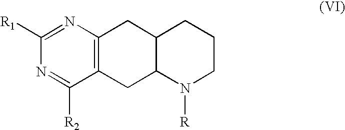

or stereoisomers thereof of formula (VIa)

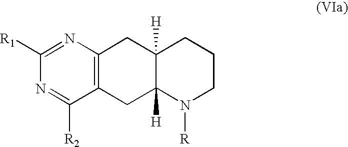

or pharmaceutically acceptable salts thereof, wherein: R is C1-C3 alkyl or allyl, R1 is NH2, NHR3, NR4 R5 and R2 is H, CH3, Cl or Br wherein R3 is methyl, ethyl or n-propyl, C1-C3 alkyl-CO, phenyl-CO or substituted phenyl-CO wherein said substituents are 1 or 2 members of the group: Cl, F, Br, CH3, C2H5, CH3 O, C2H5 O and CF3; R4 and R5 are individually methyl, ethyl or n-propyl and pharmaceutically-acceptable acid addition salts thereof.

Compounds represented by formula (VI) are generically or specifically disclosed in U.S. Pat. Nos. 4,521,421 and 4,501,890, and are generically referred to as trans-(±)-substituted-5,5a,6,7,8,9-,9a, 10-octahydropyrimido[4,5-g]quinolines. These compounds can exist as two racemates, ordinarily denominated as the trans-(±) racemate and the cis-(±) racemate. The two racemates in question are the trans-(±)-racemate-trans-(±)-2-amino-6-alkyl or allyl-4-permissibly-substituted-5,5a,6,7,8,9,9a, 10-octahydropyrimido[4,5-g ]quinoline- and the corresponding cis-(±)-racemate. The trans-(−)-enantiomer (VIa) is one of the two stereoisomers represented by (VI) and is preferred for the present invention. Specifically preferred compounds include, for example, trans-(±.)-2-amino-6-n-propyl-5,5a,6,7,8,9,9a,10-octahydropyrimido[4,5-g]quinoline, trans-(−)-2-amino-6-n-propyl-5,5a,6,7,8,9,9a,10-octahydropyrimido[4,5-g]quinoline, and trans-(−)-2-amino-6-n-propyl-5,5a,6,7,8,9,9a,10-octahydropyrimido[4,5-g]quinoline dihydrochloride.

An especially preferred compound of formula (VI) is quinelorane or pharmaceutically acceptable salts thereof. Quinelorane, or pyrido[2,3-g]quinazolin-2-amine, 5,5a,6,7,8,9,9a,10-octahydro-6-propyl-, (5aR,9aR)-(9CI) or pyrido[2,3-g]quinazolin-2-amine, 5,5a,6,7,8,9,9a,10-octahydro-6-propyl-,(5aR-trans)-, can be structurally represented by formula (VIb) below.

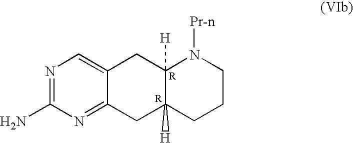

The compounds of formula (VI) and pharmaceutically acceptable salts thereof can be prepared according to the procedures set forth in U.S. Pat. Nos. 4,501,890 and 4,521,421. Information on the pharmaceutical formulations for these compounds, as well as their use and administration for treating Parkinson's syndrome, sexual dysfunction, depression, hypertension, and elevated prolactin levels, is described in U.S. Pat. Nos. 4,501,890 and 4,521,421, both incorporated herein by reference.

The compounds of formula (VI) and their pharmaceutically acceptable salts can be prepared in any conventional dosage form such as a capsule, tablet, chewable tablet, powder, transdermal patch, suspension or solution. Route of administration for these compounds in the known appropriate pharmaceutical dosage form for a given route of administration includes oral, sublingual, transdermal, and parenteral route, with oral being the preferred route.

The therapeutic effective amount of the compounds of formula (VI) in the present invention is generally from about 0.1 to about 100 mcg/kg/dose by oral administration and from about 0.25 to about 25 mcg/kg/dose by parenteral administration. The oral dosage is typically given 3-4 times per day. The exact dose to be administered and the dose frequency should be easily determined by the attending physician or clinician.

The formulations for the compounds in the present invention may conveniently be presented in unit dosage form and may be prepared by conventional pharmaceutical techniques known in the art. Such techniques include the step of bringing into association the active ingredient and pharmaceutical carriers or excipients. In general, the formulations are prepared by uniformly and intimately bringing into association the active ingredient with liquid carriers or finely divided solid carriers or both, and then, if necessary, shaping the product.

The compounds and their pharmaceutically acceptable salts which are active when given orally can be formulated as liquids, for example syrups, suspensions or emulsions, tablets, capsules and lozenges.

A liquid formulation will generally consist of a suspension or solution of the compound or pharmaceutically acceptable salt in a suitable liquid carrier(s), for example, ethanol, glycerine, non-aqueous solvent, for example polyethylene glycol, oils, or water with a suspending agent, preservative, flavoring or coloring agent.

A formulation in the form of a tablet can be prepared using any suitable pharmaceutical carrier(s) routinely used for preparing solid formulations. Examples of such carriers include magnesium stearate, starch, lactose, sucrose and cellulose.

A formulation in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

The compounds and their pharmaceutically acceptable salts which are active when administered parenterally (i.e. by injection or infusion) can be formulated as solutions or suspensions. A formulation for parenteral administration may contain anti-oxidants, buffers, bacteriostats, and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in freeze-dried (lyophilized) conditions requiring only the addition of the sterile liquid carrier, for example, water for injections, immediately prior to use. Extemporaneous injection solutions and suspensions may be prepared from sterile powders, granules, and tablets of the kind previously described.

A typical suppository formulation comprises a compound described above or a pharmaceutically acceptable salt thereof which is active when administered in this way, with a binding and/or lubricating agent such as polymeric glycols, gelatins or cocoa butter or other low melting vegetable or synthetic waxes or fats.

Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the active ingredient to be administered in a pharmaceutical acceptable carrier. Topical delivery systems include a transdermal patch and a transdermal gel containing the ingredient to be administered.

Formulations suitable for nasal administration, wherein the carrier is a solid, include a coarse powder having a particle size, for example, in the range of 20 to 500 microns, which is administered in the manner in which snuff is administered, i.e., by rapid inhalation through the nasal passage from a container of the powder held close up to the nose. Suitable formulations for nasal administration, wherein the carrier is a liquid, as for example, a nasal spray or nasal drops, include aqueous or oily solutions of the active ingredient.

The exact dosage for a given compound described above for treating symptoms of FMS or CFS of the present invention will depend on factors such as route of administration, the dosage form, the particular condition being treated, the severity of the condition being treated, the age, weight, general physical condition of the particular patient, other medication the individual may be taking, and should be easily determined by a skilled physician with experience in prescribing biologically active drugs designed to modulate central nervous system, movement and related psychological and physiological disorders of the type described here. Patients with milder forms of FMS and CFS would be expected to need less drug, patients with more severe forms of the disease may be expected to need more drug.

It will be apparent to one of ordinary skill in the art that many changes and modifications can be made in the invention without departing from the spirit or scope of the invention.

1. A method of treating the symptoms of fibromyalgia syndrome and chronic fatigue syndrome, comprising administering to a patient suffering fibromyalgia syndrome or chronic fatigue syndrome an effective amount of a compound selected from the group consisting of apomorphine, bromocriptine, pergolide, ropinirole, one of the octahydropyrazolo[3,4-g]quinolines, and one of the trans-(±)-substituted-5,5a,6,7,8,9,9a,10-octahydropyrimido[4,5-g]quinolines, or a pharmaceutically acceptable salt of any said compound.

2. The method of claim 1 wherein said compound is apomorphine or a pharmaceutically acceptable salt thereof.

3. The method of claim 2 wherein the effective amount of apomorphine or pharmaceutically acceptable salt thereof is from about 0.25 to about 12 mg/day.

4. The method of claim 1 wherein said compound is bromocriptine or a pharmaceutically acceptable salt thereof.

5. The method of claim 4 wherein the effective amount of bromocriptine or a pharmaceutically acceptable salt thereof is from about 2.5 to about 15 mg/day.

6. The method of claim 1 wherein said compound is pergolide or a pharmaceutically acceptable salt thereof.

7. The method of claim 6 wherein the effective amount of pergolide is from about 0.75 to about 5 mg/day.

8. The method of claim 1 wherein said compound is ropinirole or a pharmaceutically acceptable salt thereof.

9. The method of claim 8 wherein the effective amount of ropinirole or a pharmaceutically acceptable salt thereof is from about 0.25 to about 25 mg/day.

10. The method of claim 1 wherein said octahydropyrazolo[3,4-g]quinoline is a trans-(±)stereoisomer existing as a tautomeric pair of the formula 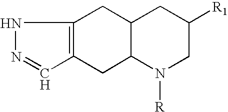
and formula 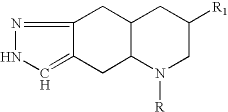
wherein:
R is H, CN, C1-C3 alkyl, allyl or benzyl and
R1 is H, COOH, COO(C1-C3) alkyl or CH2 X wherein X is CN, Cl, I, Br, OH, OCH3, SCH3, SO2 CH3, OSO2--(C1-C3)-- alkyl, O--SO2--tolyl, OSO2-phenyl or CONH2, or a pharmaceutically-acceptable salt thereof.

11. The method of claim 10 wherein R is C1-C3 alkyl or allyl and R1 is H or CH2 X wherein X is CN, CONH2, SCH3, OCH3 or SO2 CH3.

12. The method of claim 10 wherein R is H, CN or benzyl and R1 is H.

13. The method of claim 10 wherein R is C1-C3 alkyl or allyl and R1 is COO(C1-C3) alkyl, or CH2 X wherein X is Br, I, Cl, OH, OSO2--(C1-C3) alkyl, OSO2-tolyl or OSO2-phenyl.

14. The method of claim 10 wherein said octahydropyrazolo[3,4-g]quinoline is trans-(±)-5-n-propyl-4,4a,5,6,7,8,8a,9-octahydro-1H(and 2H)-pyrazolo[3,4-g]quinoline or dihydrochloride salts tereof.

15. The method of claim 10 wherein said octahydropyrazolo[3,4-g]quinoline is trans-(±)-5-n-propyl-7-methylmercaptomethyl-4,4a,5,6,7,8,8a,9-octahydro-1H(and 2H)-pyrazolo[3,4-g]quinolin or dihydrochloride salts thereof.

16. The method of claim 10 wherein said octahydropyrazolo[3,4-g]quinoline is trans-(±)-5-(C1-C3)alkyl (or allyl)-7-(C1-C3)alkoxy carbonyl-4,4a,5,6,7,8,8a,9-octahydro-1H(and 2H)-pyrazolo[3,4-g]quinoline.

17. The method of claim 10 wherein said octahydropyrazolo[3,4-g]quinoline is trans-(±)-5-n-propyl-7-ethoxycarbonyl-4,4a,5,6,7,8,8a,9-octahydro-1H(and 2H)-pyrazolo[3,4-g]quinoline.

18. The method of claim 10 wherein said octahydropyrazolo[3,4-g]quinoline is trans-(±)-5-methyl-7-ethoxycarbonyl-4,4a,5,6,7,8,8a,9-octahydro-1H(and 2H)-pyrazolo[3,4-g]quinoline.

19. The method of claim 10 wherein said octahydropyrazolo[3,4-g]quinoline is a trans-(±)stereoisomer existing as a tautomeric pair of the formula 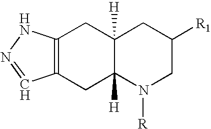
and formula 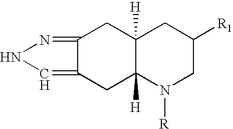
wherein R is C1-C3 alkyl or allyl, and R1 is CH2 X wherein X is CN, CONH2, SCH3, SO2 CH3 and OCH3.
